(19) United States
(12) Patent Application Publication
(10) Pub. No.: US 2025/0069240 A1
(43) Pub. Date: Feb. 27, 2025

(54) SYSTEM AND METHOD FOR AUTOMATIC REGISTRATION OF MEDICAL IMAGES

(71) Applicant: ANNALISE-AI PTY LTD, Sydney, New South Wales (AU)

(72) Inventors: Jarrel SEAH, Sydney, New South Wales (AU); Andrew JOHNSON, Sydney, New South Wales (AU); Dang-Dinh-Ang TRAN, Sydney, New South Wales (AU)

(21) Appl. No.: 18/723,293

(22) PCT Filed: Dec. 19, 2022
(86) PCT No.: PCT/AU2022/051535

§ 371 Date: Jun. 23, 2024

(30) Foreign Application Priority Data
Dec. 23, 2021 (AU) 2021904226

(51) Int. Cl. G06T 7/30; G06V 10/75; G16H 30/40

(52) U.S. Cl. CPC G06T 7/30; G06V 10/751; G16H 30/40

(57) ABSTRACT

In an implementation, a system for registering at least one medical image is provided, the system including a standardization component that may initially adjust the medical image. The system provides a deep learning model that receives the at least one medical image from the standardization component, the deep learning model generating an 3D transform matrix for the at least one medical image relative to a template image based on trained machine learning logic and an interpolation component that receives the 3D transform matrix from the deep learning model and receives the at least one medical image, the interpolation component registering the at least one medical image in three-dimensional space based on the 3D transform matrix.

### CROSS-REFERENCE TO RELATED APPLICATIONS

The present application claims priority from Australian Provisional Patent Application 2021904226 filed on 23 Dec. 2021, the contents of which are incorporated herein by reference in their entirety.

### TECHNICAL FIELD

The various aspects and embodiments described herein generally relate to automatic registration of medical images using one or more deep learning models.

### BACKGROUND

Identifying abnormalities and diseases in medical images, whether by doctors or by artificial intelligence, is much easier if the medical images are aligned to common orientations or a common reference frame. With this type of alignment, each different image of the foot or head of patients, for example, is oriented the same way each time. Simple rotation based solely on fixed registration points or added markers can be imprecise and leave artefacts particularly in 3D volume images. Therefore, automated preparation of aligned images is widely needed in the medical industry.

Some advanced normalization tools (A Python implementation, ANTsPy found online at: ANTsPy https://antspy.readthedocs.io/en/latest/) can reduce the distortions involved with a rotation, translation and resizing based on added image markers. ANTs are used for medical image registration without added markers. These normalization tools, however, consume extensive computing resources as they brute force the alignment of the images. In other words, these normalization tools require execution of iterative refinement algorithms which require a large number of steps or processes to converge on a registered image. Specifically, the alignment of various shapes in the images are shifted this way and that with optimization logic controlling the next move of the image shift. The optimization logic works by applying small updates to the orientation of an image, then looking at the level of fitness of the image to the template, then making another update to the orientation based on that fit or error. Such optimization requires hundreds or thousands of passes (image shifts) to find alignment and can take a least two minutes per medical study to successfully perform image registration. Accordingly, such alignment cannot be done quickly using conventional computers.

For more background on artificial intelligence and deep learning see U.S. Patent Publication 2018/0053114, U.S. Pat. Nos. 8,930,178, 10,043,516, 10,366,168, 10,616,199, U.S. Patent Publication 2018/0165604, and U.S. Patent Publication 2020/0082270, the disclosures of all of which are herein incorporated by reference.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

The following presents a simplified summary relating to one or more aspects and/or embodiments disclosed herein. As such, the following summary should not be considered an extensive overview relating to all contemplated aspects and/or embodiments, nor should the following summary be regarded to identify key or critical elements relating to all contemplated aspects and/or embodiments or to delineate the scope associated with any particular aspect and/or embodiment. Accordingly, the following summary has the sole purpose to present certain concepts relating to one or more aspects and/or embodiments relating to the mechanisms disclosed herein in a simplified form to precede the detailed description presented below.

It is, therefore, an object of at least one implementation of the invention to provide a faster image registration process and system for unregistered medical images, to address these present problems. Preferably, the process and system provide for a single step or pass through a trained AI model to perform image registration. In the examples, the process and system provide more reliable image registration.

In an implementation, a system for analyzing and/or registering at least one unregistered medical image is disclosed. The system includes a deep learning model generating a three-dimensional (3D) transformation matrix for an unregistered medical image provided as an input, the 3D transformation matrix being generated based on trained machine learning logic of the deep learning model. The 3D transformation matrix includes parameters that rotate and/or translate points in the unregistered medical image into a registered orientation.

The registration system includes an interpolation component configured to receive the predicted 3D transformation matrix from the deep learning model; receive the at least one unregistered medical image; and apply the received 3D transformation matrix in three dimensions to rotate and/or translation vectors representing the points [x, y, z, 1] of the at least one unregistered medical image in three-dimensional (3D) space to generate at least one registered image corresponding to the at least one unregistered medical image.

The deep learning model of the system is configured to generate six predictions for the 3D transformation matrix including three rotations around each point [x, y, z]; and three translations around each point [x, y, z]. The predicted 3D transformation matrix is a 4×4 matrix or a rigid transform matrix or an affine transform. The predicted 3D transformation matrix is a rigid transform that includes a first set of parameters and a second set of parameters for each of the at least one medical image, wherein the first set of parameters define a rotation for each of the at least one medical image and the second set of parameters define a translation for each of the at least one medical image.

In an implementation, the system includes a test component that performs a mutual information test comparing the at least one medical image registered in three-dimensional space to the template image, and the test component outputs a result of the mutual information test. If the result is below a threshold, the at least one medical image registered in three-dimensional space is rejected or flagged. The at least one medical image is a three-dimensional volume of pixels, and the 3D transformation matrix is calculated for the at least one medical image to map the at least one medical image to real world coordinates for transformation by the interpolation component.

In an implementation, the standardization component that receives the at least one medical image and analyzes the at least one medical image to validate criteria, the standardization component adjusting the at least one medical image if the criteria are not met. The at least one medical image is a raw image before processing by the standardization component, and a standardized raw image is input to the interpolation component. The standardization component outputs the standardized raw image as the at least one medical image to the deep learning component. Indeed, only the standardized raw image or a raw image may be input to the deep learning module or the standardized raw image and the template may be input to the deep learning module. After the trained machine learning logic is trained, learning from the at least one medical image input to the deep learning component may be disabled.

The deep learning model may include at least two deep learning models, each of the at least two deep learning models being trained on different training data or each of the at least two deep learning models being weighted differently. The deep learning model is trained based on a data set of medical images that are manually aligned with a template image, and the template image is a specific medical image oriented at a reference position. The deep learning model may be trained based on a data set of negative correlation data.

A method for registering at least one medical image, the method including receiving a raw medical image; inputting the raw medical image into a deep learning model; and generating a 3D transformation matrix for the raw medical image based on trained machine learning logic, wherein the 3D transformation matrix includes rotation and/or shift parameters that rotate and/or translate points in the raw medical image into a registered orientation. The method includes outputting the 3D transformation matrix to an interpolation component; and registering the raw medical image, via the interpolation component, based on the 3D transformation matrix. The registering includes receiving the predicted 3D transformation matrix from the deep learning model; receiving the at least one unregistered medical image; and applying the received 3D transformation matrix in three dimensions to rotate and/or shift vectors representing the points [x, y, z, 1] of the at least one unregistered medical image in three-dimensional (3D) space to generate at least one registered image corresponding to the at least one unregistered medical image.

A standard viewing position and scale can improve the reliability of predictions generated by AI models trained on registered images to assist radiologists in detecting abnormalities and diseases present in medical images, and can improve radiologists' interpretation of registered images rather than unregistered medical images.

Other objects and advantages associated with the aspects and embodiments disclosed herein will be apparent to those skilled in the art based on the accompanying drawings and detailed description.

### BRIEF DESCRIPTION OF THE DRAWINGS

A more complete appreciation of the various aspects and embodiments described herein and many attendant advantages thereof will be readily obtained as the same becomes better understood by reference to the following detailed description when considered in connection with the accompanying drawings which are presented solely for illustration and not limitation, and in which:

FIG. 1 is a system diagram of a training and the image registration system according to an implementation;

FIG. 2 is a system diagram of the image registration system according to an implementation;

FIG. 3 is a flow diagram of the image registration system according to an implementation;

FIG. 4 is a process for image registration according to an implementation;

FIG. 5 is a block diagram of the deep learning component according to an implementation;

FIG. 6 is a flow diagram for a training process according to an implementation;

FIG. 7 is a process for training a machine learning model according to an implementation;

FIG. 8 is a bock diagram of example hardware for the system according to an implementation; and

FIG. 9 is an example registration performed by the method or system according to an implementation.

### DETAILED DESCRIPTION

Various aspects and embodiments are disclosed in the following description and related drawings to show specific examples relating to exemplary aspects and embodiments. Alternate aspects and embodiments will be apparent to those skilled in the pertinent art upon reading this disclosure, and may be constructed and practiced without departing from the scope or spirit of the disclosure. Additionally, well-known elements will not be described in detail or may be omitted so as to not obscure the relevant details of the aspects and embodiments disclosed herein.

The word “exemplary” is used herein to mean “serving as an example, instance, or illustration.” Any embodiment described herein as “exemplary” is not necessarily to be construed as preferred or advantageous over other embodiments. Likewise, the term “embodiments” does not require that all embodiments include the discussed feature, advantage, or mode of operation.

The terminology used herein describes particular embodiments only and should not be construed to limit any embodiments disclosed herein. As used herein, the singular forms “a,” “an,” and “the” are intended to include the plural forms as well, unless the context clearly indicates otherwise. Those skilled in the art will further understand that the terms “comprises,” “comprising,” “includes,” and/or “including,” as used herein, specify the presence of stated features, integers, steps, operations, elements, and/or components, but do not preclude the presence or addition of one or more other features, integers, steps, operations, elements, components, and/or groups thereof.

Further, various aspects and/or embodiments may be described in terms of sequences of actions to be performed by, for example, elements of a computing device. Those skilled in the art will recognize that various actions described herein can be performed by specific circuits (e.g., an application specific integrated circuit (ASIC)), by program instructions being executed by one or more processors, or by a combination of both. Additionally, these sequences of actions described herein can be considered to be embodied entirely within any form of non-transitory computer-readable medium having stored thereon a corresponding set of computer instructions that upon execution would cause an associated processor to perform the functionality described herein. Thus, the various aspects described herein may be embodied in a number of different forms, all of which have been contemplated to be within the scope of the claimed subject matter. In addition, for each of the aspects described herein, the corresponding form of any such aspects may be described herein as, for example, “logic configured to” and/or other structural components configured to perform the described action.

Those skilled in the art will appreciate that information and signals may be represented using any of a variety of different technologies and techniques. For example, data, instructions, transmissions, commands, information, signals, bits, symbols, and chips that may be referenced throughout the above description may be represented by voltages, currents, electromagnetic waves, magnetic fields or particles, optical fields or particles, or any combination thereof.

Further, those skilled in the art will appreciate that the various illustrative logical blocks, modules, circuits, and algorithm steps described in connection with the aspects disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted to depart from the scope of the various aspects and embodiments described herein.

The various illustrative logical blocks, modules, and circuits described in connection with the aspects disclosed herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, a plurality of microprocessors, one or more microprocessors in conjunction with a DSP core, or any other such configuration).

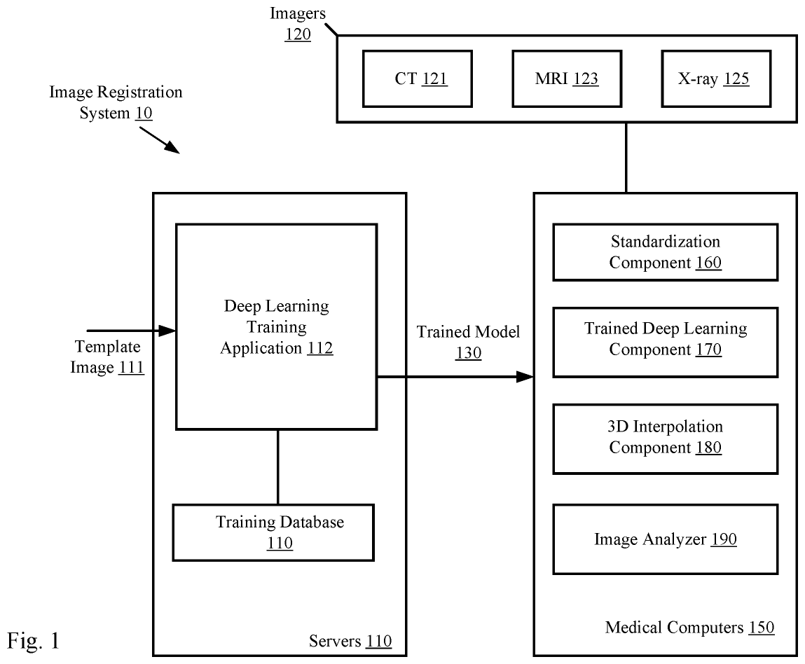

FIG. 1 depicts an image registration system 10 that trains a deep learning model (‘registration model’) on hardware separate from the hardware performing the image registration. The training of the registration model is performed using computed tomography brain (CTB) images that are randomly shifted. The specific random shift that is applied function as labels. Data is generated synthetically due to the random shift. The registration model may comprise a deep convolutional neural network (CNN) component. Due to regulations and the lack of availability of high performance compute capabilities in many countries this separation may be present. Nevertheless, the training hardware, servers 110, is certainly capable of registering an image just as the medical computers 150 can have a copy of the trained deep learning model to generate medical prediction(s) used to register the image provided as an input to the trained deep learning model, at least because the training on servers 110 involves millions of registrations like those performed on the medical computers 150. Accordingly, in some implementations, the servers 110 could also perform inferencing using the trained deep learning model and generate the medical prediction(s) as well as register the medical images before ultimately delivering the medical images to doctors. Likewise, medical computers 150 may be used to train the deep learning model as well in some implementations. Such implementations, however, may require a significant amount of time compared to using cloud computing with more powerful computing resources. Virtual separation (e.g., separate virtual machines, separate virtual subnets, etc.) rather than physical separation may be applied to a combined solution.

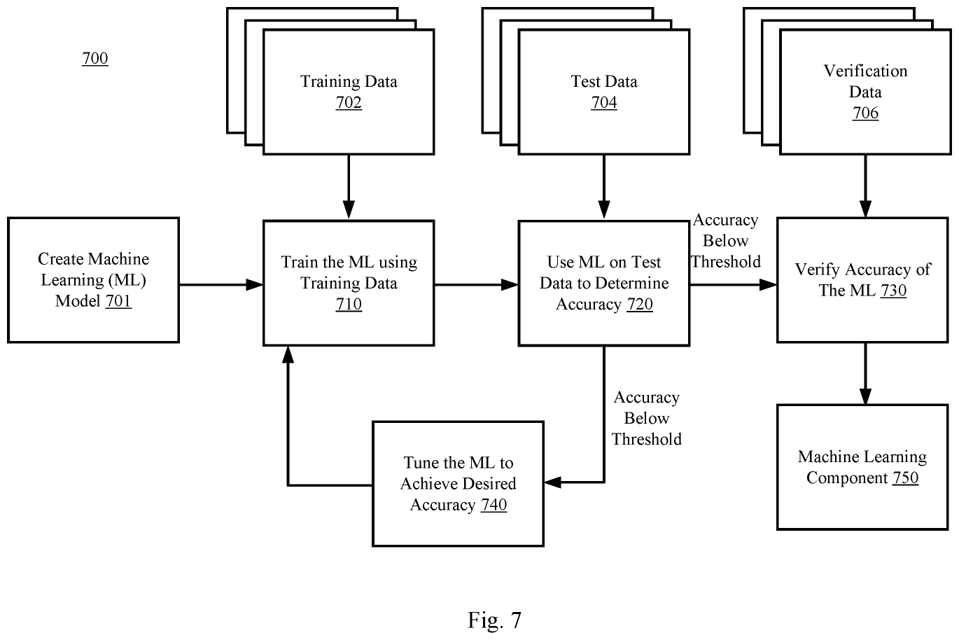

The training servers 110 may receive a dataset containing a plurality of template images 111 which may have a desired or predetermined alignment. The template image 111 may be a 3D volume or a series of layer slices of a 3D volume or a 2D image. For example, a template image 111 for a chest X-ray image may be a 2D image whereas a template image 111 for a computer tomography (CT) brain image may be a 3D volume. The template image 111 may be received by the deep learning training application 112 which is a testing environment arranged to repeatedly register images to the template image 111 and test the result of that registration before using the feedback from that test to update and improve the machine learning being trained. This process is described in more detail with respect to FIG. 7.

The deep learning training application 112 may retrieve images from interrogating a training database 110 which may contain, for example, at least 200,000 example images, which can be registered by applying transformations using the input template image 111. A validation database may include expertly curated or known medical images that correspond to the test images to check the accuracy of the deep learning. Data may be generated by taking existing registered images (e.g. that may have been registered using ANTs) and randomly rotating the volume of these existing registered images and then training the model to correct the random rotation applied. For other examples, the deep learning training application 112 may retrieve expertly curated or known registered medical images and then apply distortions to improve the robustness of the deep learning model in the event of imperfect data such as minor perturbations. The training database 110 may store other information for training including verification data, mutual information test data, additional template images 111, and negative correlation information. The applications of each piece of additional information to the training process will be described in more detail below.

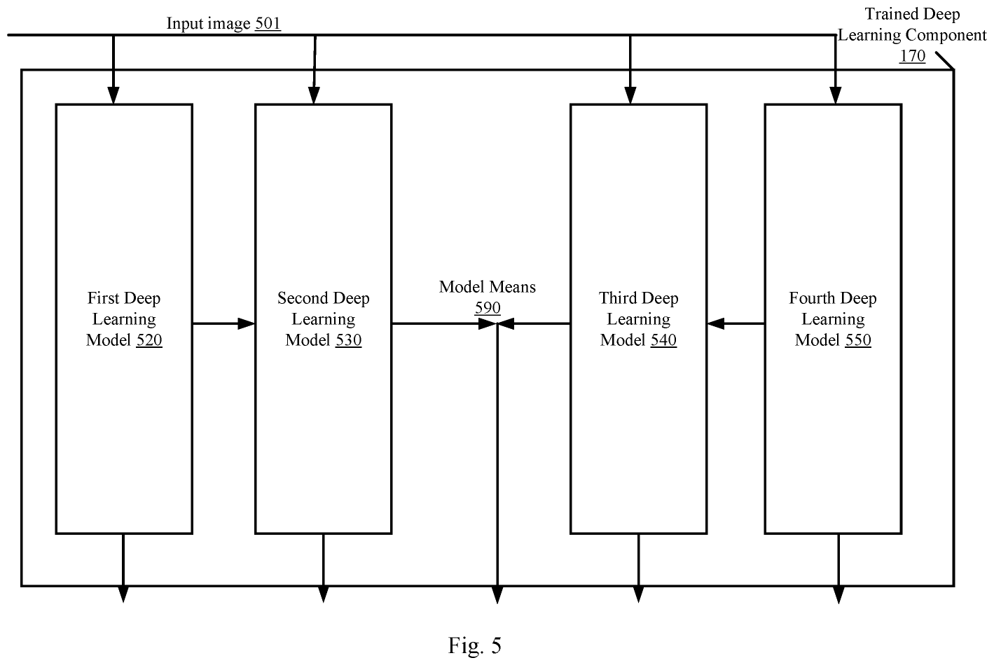

The deep learning training application 112 may include options and parameters which are tunable to focus the registration model 130 on particular accuracy issues. For example, a registration model 130 may be tuned to properly register images in various error scenarios (e.g., information dropout, images with artefacts, motion distortions, etc.), another registration model may be tuned to properly register images with certain diseases (e.g., major trauma), another registration model may be tuned using negative correlation information as described further below. Each of these separately trained models may then be applied to an unseen (new) image from an actual clinical workflow on patients and obtain more accurate results than a single, unspecialized, trained model. Indeed, the trained model 130 passed to the medical computers 150 may include two or more models ensembled into a fully-trained model as illustrated in FIG. 5. In an implementation, the registration models may not be separately trained such that there is only a single registration model. The single model can be improved by up-weighting frequencies in the training set using oversampling or loss weighting. In an implementation, the registration models are not ensembled. In an implementation, the registration models may be ensembled.

The medical computers 150 may receive the trained model 130 and may load and deploy that trained model 130 into the trained deep learning component 170. The medical computers 150 may be connected (directly or indirectly) to various imagers 120 such as computed tomography (CT) imager 121, magnetic resonance imager 123, X-ray imager 125 and/or other imagers (e.g., positron emission tomography (PET), infrared (IR)). The medical computers 150 may be co-located with such imagers or may be co-located with doctors (including remotely) such that the connection between the imagers 120 and medical computers 150 may be over the internet, ethernet, a local area network, fiber optics, wireless network, or the like. The raw medical images (e.g. DICOM files prior to any correction/standardization or registration) from the imagers 120 are unregistered and may first be received by the one or more medical computers 150 and are provided as an input for the standardization component 160.

There may be a preprocessing step prior to passing the image/volume into the registration model. During this step, the shape of an image volume and a voxel spacing is updated and standardized. The standardizing component 160 may adjust or determine a center or axis of the images or volumes, adjust the size of or scale the image (dimensions in pixels, volumetric pixel or voxels), and determine a spacing, origin, direction, and shape of the unregistered image. The standardizing component 160 standardizes the images to a common shape and voxel spacing, focusing on the head region of a CTB. The standardization component 160 may perform image analysis on the images for error detection. The system may calculate the real-world parameters of the raw, unregistered image based on features in the unregistered image so as to enable these parameters to be adjusted by the image registration system. At the time that the unregistered image is provided as an input, the image registration system may not have information on the orientation of each raw, unregistered image or only a portion of such information is supplied by the imager 120. In an example, image orientation data is stored in the DICOM metadata. The 3D spatial tensor is created from information within the DICOM headers In an implementation, the standardization component 160 may calculate the initial positioning of the raw, unregistered image based on features and symmetry and generate a 3D spatial tensor (as a data structure containing the position and orientation vectors) that is provided as an output of the standardization component 160. The calculations, definition, or analysis of the position may be performed by a separate trained machine learning model. The correction component may at least adjust the size of the received image or volume by re-sampling the image to arrive at the predetermined size.

The standardized unregistered image with positioning information may be transmitted to the trained deep learning component 170. The deep learning component 170 may receive this unregistered image as the only input for the deep learning based image registration process. In an implementation, the deep learning component 170 may select an appropriate model (any one from a plurality of trained AI models) if different models are available. For example, if the unregistered image provided as an input to the deep learning component 170 is a brain CT image, then the deep learning component 170 selects and deploys a trained deep learning model that was trained to register unregistered brain CT images. The trained deep learning component 170 may then predict (by calculating), by executing the trained machine learning logic, an affine transform to rotate and/or translate the unregistered medical image. The affine/rigid transform predicted by the deep learning component 170 and the standardized, registered image from the correction component 160 are then transmitted as inputs to the 3D interpolation component 180. The 3D interpolation component 180 then applies the predicted affine/rigid transform by re-sampling, via interpolation, the unregistered medical image into the new orientation as defined by the affine transform and the unregistered medical images' 3D spatial tensor. The re-sampled image is then the registered image with an orientation matching that of the template image 111. This re-sampled image is output from the 3D interpolation component 180 and is considered the registered image or volume. The registered image or volume may be transmitted further to a display for a physician to review, be included in a training dataset to train an AI model to classify and segment medical images, or be sent to image analyzer 190 comprising a deep learning model for predicting the presence of diseases and injuries in medical images or localizing or segmenting detected diseases and injuries in medical images.

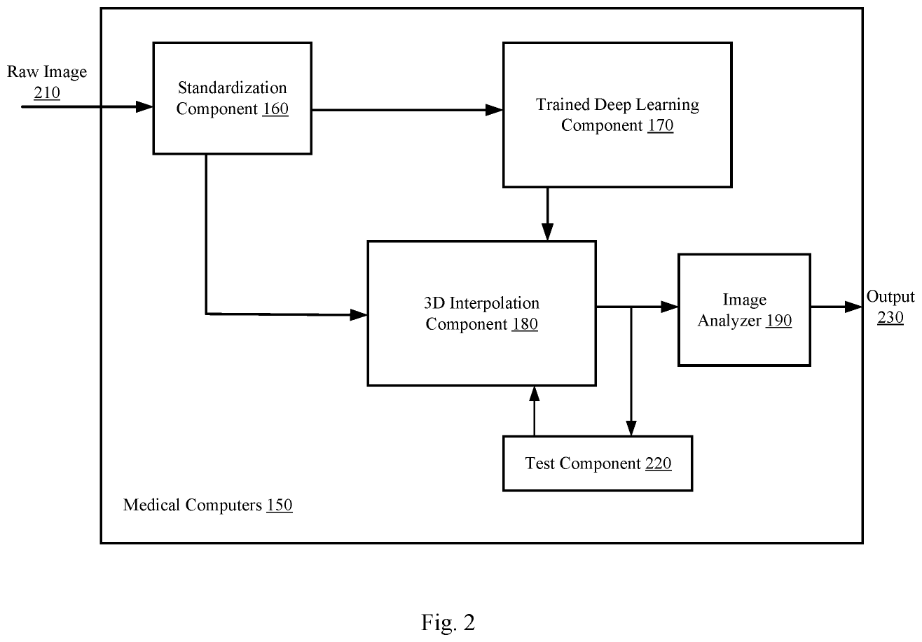

In FIG. 2 the flow of information between components as described above within the medical computers 150 is illustrated in more detail. Initially a raw image 210 from the imagers 120 is input (e.g., a Digital Imaging and Communications in Medicine (DICOM file)) along with any metadata stored in the file's header describing the imager's 120 type, settings and parameters. The standardization component 160 receives the raw, unregistered image 210 and calculates the position matrix for the image in 2D or 3D, as the case may be, and resizes the raw, unregistered image 210. The standardized unregistered image (if resized, for example) or the raw, unregistered image 210 may be provided to the trained deep learning component 170 which may input the image into several different models trained on different aspects of the image. First, the deep learning component 170 may determine the relevant part of the body in the image and then select a corresponding set of trained models. The trained models may be arranged or connected in an ensemble as illustrated in FIG. 5, for example. Each of the trained models for the relevant body part may receive the medical image and calculate an affine transform for image registration in parallel with each other.

After the ensemble of models (or a single model) has agreed upon an affine/rigid transform, the calculated 3D transform is output to the 3D interpolation component 180 from the deep learning component 170. The 3D interpolation component 180 also receives or retrieves the raw, unregistered image or standardized image along with the affine transform. The 3D interpolation component 180 then resamples the unregistered/standardized image based on the rigid transform (i.e., at positions defined by a affine transform). The 3D interpolation component 180 may perform such interpolation on 2D images, 3D volumes, and 2D slices of 3D volumes. The transformed image is a rigid transformation of the original with rotations or translations applied. This transformed image is the registered image corresponding to the unregistered image. The transformed image may be output to a test component 220 for validation and to an image analyzer 190 for automated detection of diseases and injuries. Additionally or alternatively, the transformed image may be output at output 230 to a physician or radiologist. The test component 220 may provide feedback, warnings or flags to the 3D interpolation component 180 if the registered image cannot be validated (e.g., is distorted).

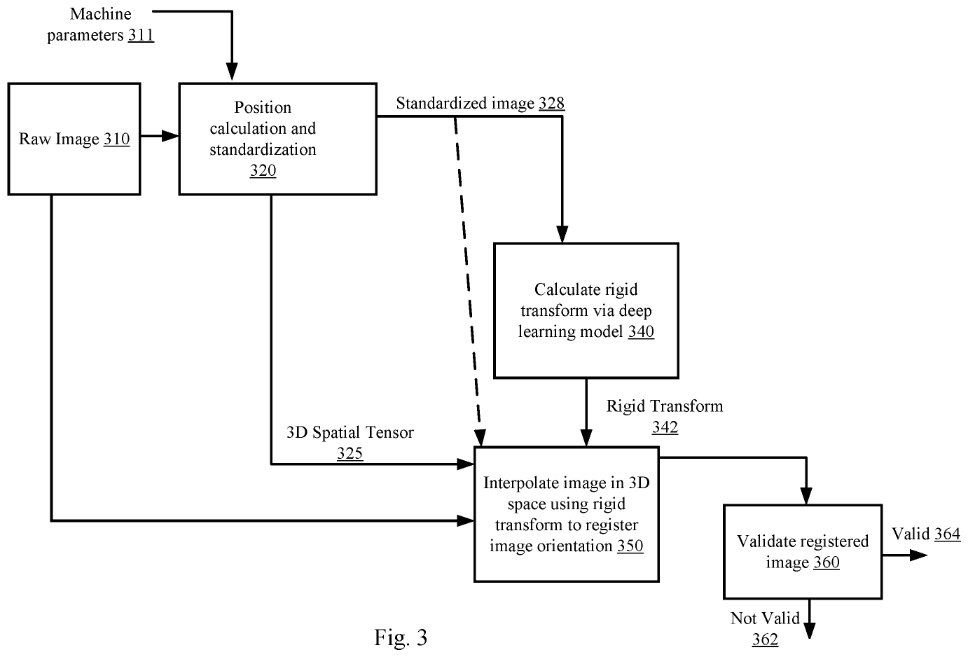

In FIG. 3 the flow of information for the registration process is illustrated in more detail. The process begins with a raw, unregistered image 310/210 which is input into the standardization component 160 to ascertain the position of the image and evaluate if any adjustment to the unregistered image 310 is needed. Specifically, the position and orientation of the unregistered image 310 in the real world may be needed in order to apply the affine transform. The position and orientation may be characterized or defined as one or more 3D spatial tensors 325 (matrix) and may be output from the correction component 160 to the 3D interpolation component 180 or stored in memory for the 3D interpolation component 180 to retrieve. If adjustments are needed to the unregistered image 310, these are also performed and a standardized unregistered image 328 is generated. For example, if the deep learning model requires a particular image size (e.g., 126×256×256 voxels) or voxel spacing, then the standardization component 160 may adjust the raw, unregistered image 310 by re-sampling the unregistered image at the smaller size.

Next the system may calculate a rigid transform (or affine transform) for the standardized unregistered image 328 in the deep learning model at step 340. This calculation, for example, may separately define a rotation matrix which may have three parameters as a vector and a translation matrix which may have three parameters as a vector that together form the rigid transform 342 for registering the image. Advantageously, the deep learning model already has the template image 111 or target orientation trained into one or more the neural network layers of the deep learning model. Thus, no template image 111 is required as an input to the deep learning model. In an example, the neural network does not consist of (or include) the template image 111 but rather the template image 111 is provided as a separate input to the neural network. This may be helpful when registering two different image series to each other rather than registering an unregistered image to a common template. The rigid transform 342, in this example, may be output to the 3D interpolation component 180 for application to the 3D spatial tensors calculated for the medical image. The rigid transform 342 may be of the form of a four by three matrix or a four by four matrix, for example. In general, the deep learning model at step 340 may generate six predictions (vectors) for the 3D transformation matrix including three rotations around each point [x, y, z] of the image; and three translations around each point [x, y, z] of the image. The 3D interpolator of 3D interpolation component 180 then re-samples the standardized unregistered image 328 (if present) or the raw, unregistered image 310 in real space using both the 3D spatial tensors 325 and the transformation matrix (affine/rigid transform) 342 to register the unregistered image orientation correctly at step 350. The re-sampled image may be output for validation to the test component 220 to validate the registered image at step 360. If not valid at step 362, the process may reset, shift to another mode, and/or signal that an error has been detected. If the registered image is valid at step 364, then the registered image is displayed and/or analyzed as described above.

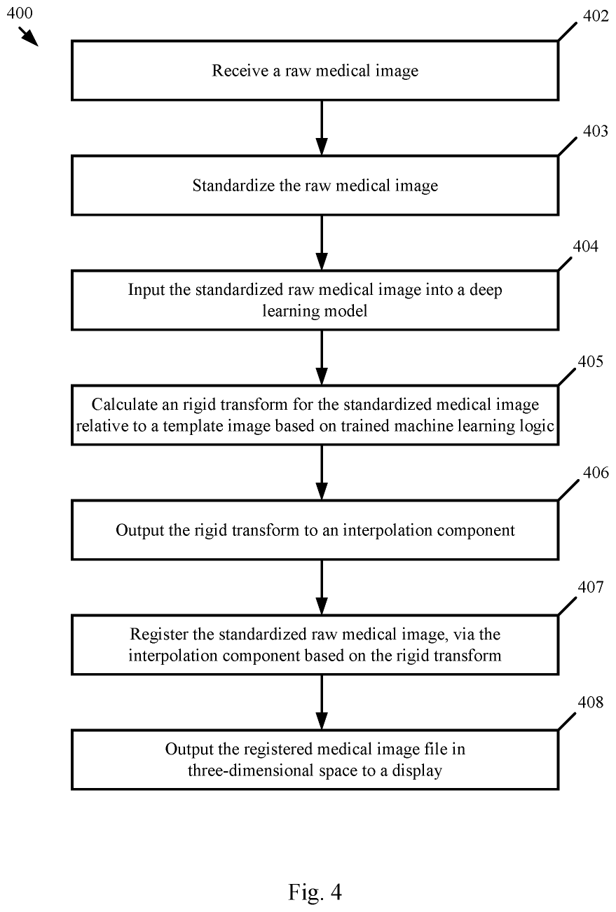

The process 400 of FIG. 4 depicts operational components and functions that may be performed by the system in an implementation. At step 402, a raw medical image is received, for example from imagers 120. At step 403, any corrections or standardizations that are needed are made to the raw, unregistered medical image. At step 404, the standardized unregistered image is input into the deep learning model that has previously been trained. At step 405, the system calculates a 3D transformation matrix (e.g., an affine or rigid transform) for the corrected unregistered medical image relative to a template image 111 based on the trained machine learning logic. Specifically, the calculation may be performed solely based on the trained machine learning logic without the template image 111 itself. The predicted 3D transformation matrix includes rotation and/or shift parameters that are predicted to rotate and/or shift points in the at least one unregistered medical image into a registered orientation. At step 406, the rigid transform 342 or, generally, a 3D transformation matrix is output to a 3D interpolation component 180 in order to be applied to the unregistered image or standardized unregistered image. Applying the received 3D transformation matrix in three dimensions may include applying rotate and/or shift vectors representing the points [x, y, z, 1] of the at least one unregistered medical image in three-dimensional (3D) space (or movement of the points) to generate at least one registered image corresponding to the at least one unregistered medical image. At step 407, the medical image is registered based on the 3D transform matrix and the calculated 3D tensor of the raw image via the 3D interpolation component 180. At step 408, the registered image is output to a display for viewing by the physician. Additional analysis may also be performed and applied upon display.

In FIG. 5, various sub-components of the trained deep learning component 170 are depicted. The deep learning component 170 intakes or receives an input image 501 that is a raw or adjusted medical image. The deep learning component 170 may determine which models apply to the particular medical image or volume (e.g., brain CT). The deep learning component 170 may then transmit the medical image or volume to one or models trained for registration of that type of image (e.g., brain CT). The first deep learning model 520, second deep learning model 530, third deep learning model 540, and fourth deep learning model 550 may execute in parallel or sequentially. For example, the first and fourth deep learning models 520/550 may execute first and output their analysis as well as an evaluation of success to the second and third deep learning models 530/540, respectively. The first and fourth deep learning models 520/550 may execute and output their analysis as well as model means 590 (e.g., average of result) to the second and third deep learning models, respectively. Each model mean of the model means 590 may be the average of the model output which may be used to compare models and for weighting. More or fewer deep learning models may be provided and operate in parallel or sequentially in much the same way.

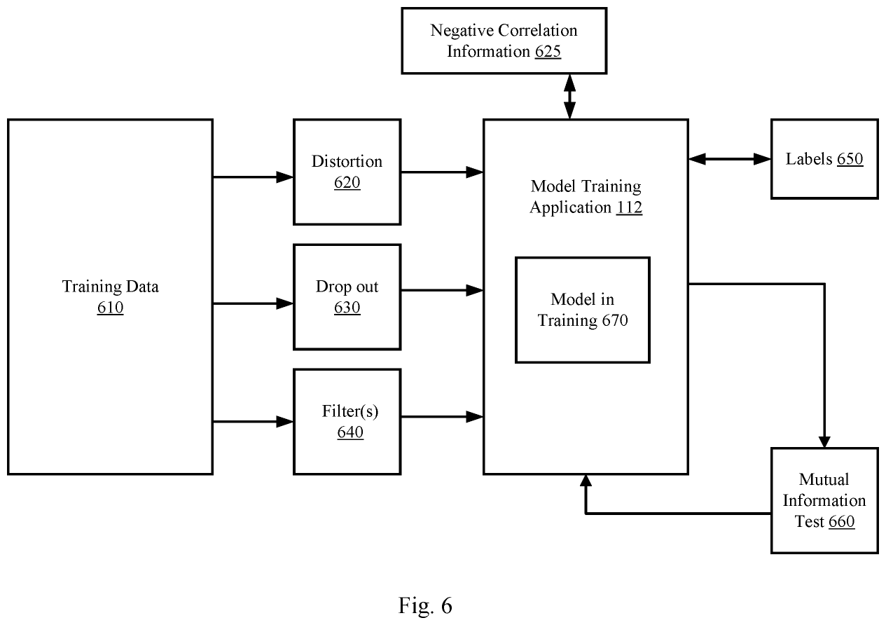

The different deep learning models may be trained separately or use different training datasets, to handle different issues as illustrated in FIG. 6. For example, the first deep learning model may be trained on medical images where an expected rotation/translation range is present but one of slices is corrupted or segments of the volume are missing. For example, the second deep learning model may be trained on medical images where an expected rotation/translation range is present but massive trauma and injury are present. For example, the third deep learning model may be trained on medical images where an expected rotation/translation range is present but minor shifts in the patient occurred during imaging. For example, the fourth deep learning model may be trained on medical images where an unexpected rotation/translation range is present. Each of the models may output a model mean 590 and a model goodness-of-fit metric for comparison. The models may then be ensembled according to various methods including weighting based on goodness-of-fit, averaging, or ranked selection, for example. Even though all the models may output an affine transform and quality metrics, the model means 590 may be compared so that a single affine transform can be selected or calculated. This single affine transform may be output separately. One or more of the outputs may be transmitted to the 3D interpolation component 180.

Each of the models 520-550 may perform windowing and down sampling as initial or intermediate steps in the calculations of the affine transform. For example, the input image may be windowed into multiple segments initially then recomposed after a series of machine logic steps (or neural network layer traversals). The recomposed image may then be down sampled and re-processed through more neural network layers or machine logic steps. Specifically, the neural network layers or machine logic steps are the elements of the model that have been trained to recognize image features and align/register the image. The neural network layers or machine logic may have incorporated the knowledge and features of the template image 111 such that it is not needed as an input. Other adjustments may also be performed in each of the models such as up sampling, segmentation, normalization, classification, dropouts, recompositions, and other image manipulation techniques that can improve performance of the neural network layers of the deep learning model or machine logic at various steps.

In FIG. 6 the information flows with respect to the training process are illustrated for various testing and training situations. The training data 610 may include properly registered medical images, unregistered medical images, the template image 111, and other expected information provided along with such images (e.g., meta data). These medical images may be altered via distortion 620 (e.g., medical implants, patient shifts, etc.), drop outs 630 where a slice or portion of the image/volume is dropped (e.g., to simulate dropped signals or data from transmission), or filters 640 which may only pass certain image types (e.g., with tumors, trauma, fluid, etc.) to the model training application 112. Accordingly, depending on the adjustment made, the developed or trained model may be adapted to function well on certain image types.

The model training application 112 may receive registered medical images whose orientation is already correctly aligned. The model training application 112 generates a random rigid transform (affine transform) that may be within a certain range. Next, the model training application 112 applies this random rigid transform to the registered medical images by re-sampling and outputs a de-registered image. This de-registered image generated by the model training application 112 is then input as training data to the model being trained 670 and the random rigid transform is output to the labels 650 as a label stored in memory. The model being trained 670 then predicts an affine transform which is compared to the stored label and used to improve the model as described in more detail in FIG. 7. The registered medical image may have had artefacts added to it as described above. Furthermore, an unregistered medical image may be input to the model along with a label for the correct adjusting transform which may have been calculated or determined previously. In either case, the model under training calculates an affine transform and is improved by comparison with the correct label.

The model training application 112 may utilize the negative correlation information 625 to ensure that the deep learning model is able to correctly classify or identify a registration orientation X not due to the presence of a given Y variable (where the presence of the Y variable is highly correlated to the registration orientation X). In other words, the negative correlation information 625 provides data or training scenarios to ensure that the deep learning model is trained with sufficient training data that has registration orientation X without the correlated variable Y and vice-versa.

After registering of an unregistered image as a way of testing the performance of the trained model on unseen/new data (e.g., test data), the registered test image may be output to a mutual information test of a mutual information test component 660. The mutual information test component 660 determines the mutual dependence of the registered image relative to the template image 111 and may provide positive or validating feedback even though the data in the images is different. That is, a large percentage of features in a brain or chest image, for example, are expected to match the template if correctly registered even if some parts are abnormal, injured or diseased. More specifically, it quantifies the amount of information in dimensionless numbers that can be obtained about one random variable or image by observing the other random variable or image. This test may be applied in training for test and validation and afterwards in the field on medical computers 150 as a quality assurance measure.

FIG. 7 is a flowchart of a process 700 to train a machine learning algorithm or deep learning model, according to some implementations. The process 700 may be performed by the one or more servers 110 of FIG. 1. The training may be applied to each deep learning model of the trained deep learning component 170, applied to machine learning models for calculating the initial 3D spatial tensor, or applied to machine learning models for identifying abnormalities, diseases and injuries as provided in the image analyzer 190.

At step 701, the machine learning algorithm (e.g., software code) may be created by one or more software designers with a number of nodes and layers to be trained. At step 710, the deep learning model (e.g. machine learning algorithm) may be trained using pre-registered training data 702. The training data 702 may be pre-registered images that have been transformed by a known transform such that the training step 710 is supplied with the de-registered image and the correct transform for comparison. For example, the training data 702 may include pre-registered or pre-aligned by humans, by brute force methods (e.g., ANTs), or a combination of both. After the deep learning model has been trained using the training data 702, the deep learning model may be tested, at step 720, using test data 704 to determine an accuracy of the deep learning model. The test data 704 may utilize a combination of unregistered medical images and mutual information tests 660 for testing using new data. For example, in the case of an alignment, the accuracy of the alignment may be determined using the test data 704 and the mutual information test component 660.

If a metric (e.g. an evaluation metric such as AUC, or mutual information) of the deep learning model does not satisfy a desired accuracy threshold (e.g., 95%, 98%, 99% accurate), then at step 740, the machine learning code may be tuned, to achieve the desired accuracy. For example, at step 740, the software designers may modify the machine learning software code to improve the accuracy of the machine learning algorithm, prune the training data, or otherwise adjust the training data and process. After the deep learning model has been tuned, at step 740, the deep learning model may be retrained, at step 710, using the pre-classified training data 702. In this way, steps 710, 720, and 740 may be repeated until the deep learning model is able to classify the test data 704 with the desired accuracy.

After determining that an accuracy of the deep learning model satisfies the desired accuracy threshold, the process may proceed to step 730 where verification data for 706 may be used to verify an accuracy of the deep learning model. After the accuracy of the deep learning model is verified, at 730, the machine learning component or logic 750, which has been trained to provide a particular level of accuracy, may be saved or stored. The trained machine learning logic 750 may then be modified to prevent further learning and exported or uploaded to the trained deep learning component 170 for use as in FIG. 5. The process 700 may be used to train any of the machine learning algorithms or logic contemplated herein. For example, in trained deep learning component 170, a first machine learning model may be used to make first calculated affine transform nnn, a second machine learning model may be used to calculate affine transform mmm, a third machine learning model may be used to calculate affine transform ppp, and so on.

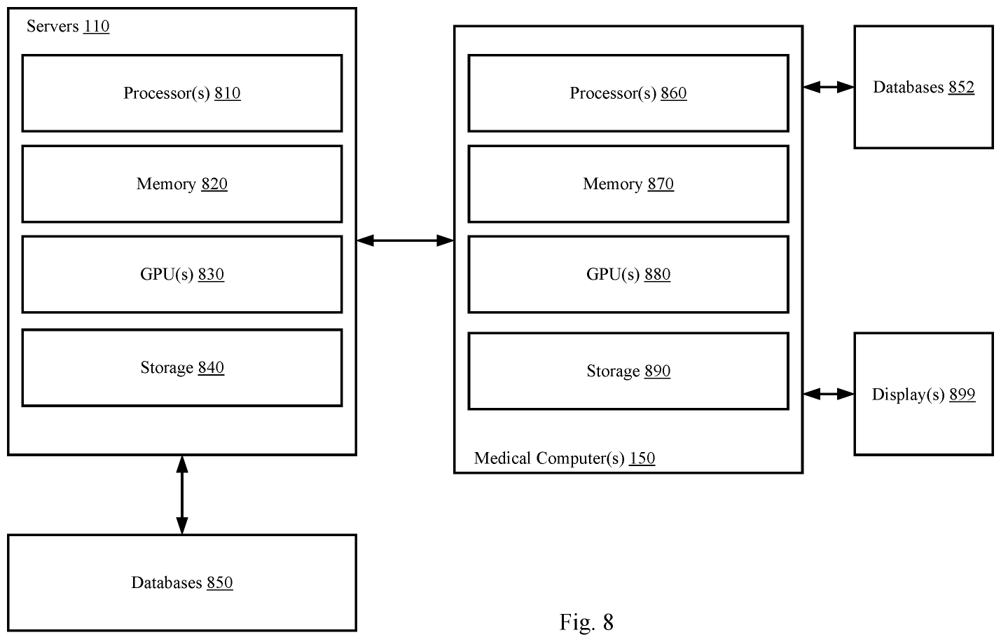

In FIG. 8 example hardware for the servers 110 and the medical computers 150 are illustrated. The servers 110 may include one or more processors 810, one or more volumes of memory 820, one or more graphic processing units (GPUs) 830, and one or more storage volumes 840. A combination of processors 810 and GPUs 830 may provide the processing power to train the machine learning or deep learning models. The memory 820 may be short-term random-access memory and storage 840 may be hard, non-volatile storage. The servers 110 may connect to one or more databases 850 which may be on separate servers and may contain the training data. The servers 110 may communicate with medical computers 150 for upload of machine learning models and the like.

The medical computers may include one or more processors 860, one or more volumes of memory 870, one or more graphic processing units (GPUs) 880, and one or more storage volumes 890. The medical computers 150 may connect to one or more displays 899 to provide information and registered images to radiologists. The medical computers 150 may connect to one or more databases which may contain patient medical images and other relevant information. The processing power for registration of the unregistered medical images and determination of the spatial tensors may be provided by processors 860 or GPUs 880 or a combination thereof.

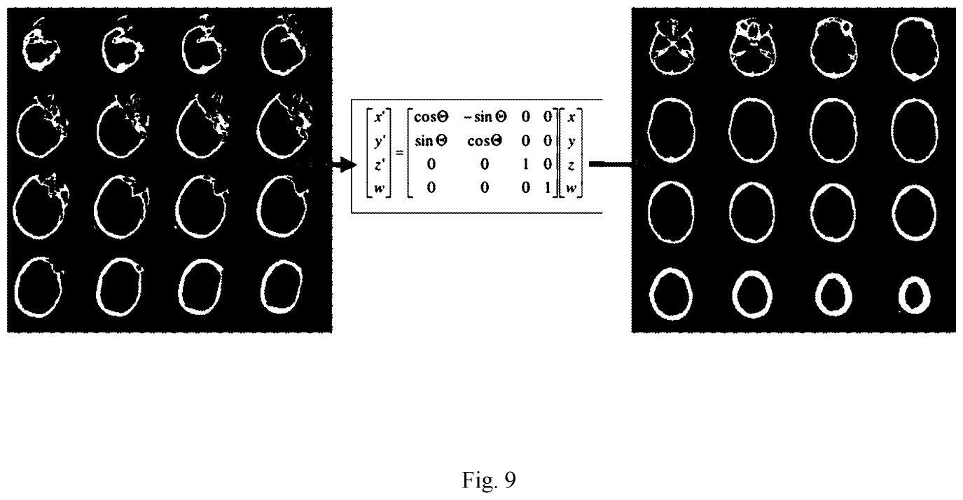

FIG. 9 illustrates an example registration of a brain CT image according to an implementation. Specifically, an unregistered volume 910 from a CT imager 121 may be provide as an input to the trained deep learning component 170 in order to predict the transformation matrix 920 (e.g. affine transform) for rotation along the z-axis, for example, may be calculated and determined. In this example, the predicted 3D transformation matrix includes a rotation vector. The predicted transformation matrix 920 is then applied in the interpolation component on the unregistered volume 910 to generate and output the registered brain CT image 930. The 3D transformation matrix may include one, two, or three rotation vectors and/or one, two, or three translation vectors. While images slices are shown here, the deep learning model may have been applied to and registered the entire 3D volume (voxel volume) and image slices are displayed or visually communicated to an end user, such as a radiologist.

Besides registration of unregistered medical images to a standardized orientation, the disclosed system and method may also (1) fix gantry tilt, (2) fix data leak from gantry tilt, (3) slice down center for left/right localization, and (4) register to an atlas (i.e. a template image 111) and be used as training data to train another AI model to localise/segment specific radiological findings present in a medical image. Specifically, once trained to recognize proper registration, the deep learning models described herein can overcome many different errors that may impair the images and would prevent registration or normalization under conventional processes. Other advantages of the above-described system for registering unregistered medical images include avoiding/minimizing many postprocessing steps where there are multiple series require registration (to each other, rather than to a common template). For example, a common postprocessing step is digital subtraction, that is, subtracting a pre-intravenous contrast CT from a post-intravenous contrast CT to generate a map of the arteries, which requires both of these CT images to be registered.

The registered images generated by the disclosed system and method are useful because they may be used as training data to train another AI model. Such an AI model can classify and segment abnormalities and diseases in a medical image provided to the AI model, to assist a radiologist in their clinical decision. Another important use of the registered images is that they can be displayed in a graphical user interface component (in original size or as a thumbnail image) such as a widget via a client application or web browser, to a radiologist for easier visual validation whether an abnormality or disease is present in a medical image. Another important use of the registered images is that volume measurement and volume analysis is possible for these images. In other words, for example, a brain lesion detected in a CT scan can be measured as 8 cm³ on the computer display using a measurement software component, since every pixel has a predefined real-world measurement (e.g. each pixel corresponds to 1 mm in length).

The methods, sequences, logic and/or algorithms described in connection with the aspects disclosed herein may be embodied directly in hardware, in a software module executed by a processor, or in a combination of the two. A software module may reside in RAM, flash memory, ROM, EPROM, EEPROM, registers, hard disk, a removable disk, a CD-ROM, or any other form of non-transitory computer-readable medium known in the art. An exemplary non-transitory computer-readable medium may be coupled to the processor such that the processor can read information from, and write information to, the non-transitory computer-readable medium. In the alternative, the non-transitory computer-readable medium may be integral to the processor. The processor and the non-transitory computer-readable medium may reside in an ASIC. The ASIC may reside in an IoT device. In the alternative, the processor and the non-transitory computer-readable medium may be discrete components in a user terminal.

In one or more exemplary aspects, the functions described herein may be implemented in hardware, software, firmware, or any combination thereof. If implemented in software, the functions may be stored on or transmitted over as one or more instructions or code on a non-transitory computer-readable medium. Computer-readable media may include storage media and/or communication media including any non-transitory medium that may facilitate transferring a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, DSL, or wireless technologies such as infrared, radio, and microwave, then the coaxial cable, fiber optic cable, twisted pair, DSL, or wireless technologies such as infrared, radio, and microwave are included in the definition of a medium. The term disk and disc, which may be used interchangeably herein, includes CD, laser disc, optical disc, DVD, floppy disk, and Blu-ray discs, which usually reproduce data magnetically and/or optically with lasers. Combinations of the above should also be included within the scope of computer-readable media.

While the foregoing disclosure shows illustrative aspects and embodiments, those skilled in the art will appreciate that various changes and modifications could be made herein without departing from the scope of the disclosure as defined by the appended claims. Furthermore, in accordance with the various illustrative aspects and embodiments described herein, those skilled in the art will appreciate that the functions, steps, and/or actions in any methods described above and/or recited in any method claims appended hereto need not be performed in any particular order. Further still, to the extent that any elements are described above or recited in the appended claims in a singular form, those skilled in the art will appreciate that singular form(s) contemplate the plural as well unless limitation to the singular form(s) is explicitly stated.

1. A system for analyzing and/or registering at least one unregistered medical image, the system comprising:
a deep learning model generating a three-dimensional (3D) transformation matrix for an unregistered medical image provided as an input, the 3D transformation matrix being generated based on trained machine learning logic of the deep learning model,
wherein the 3D transformation matrix includes parameters that rotate and/or translate points in the unregistered medical image into a registered orientation.

2. The system of claim 1, further comprising:
an interpolation component configured to:
receive the 3D transformation matrix from the deep learning model;
receive the unregistered medical image; and
apply the 3D transformation matrix in three dimensions to rotate and/or translate points of the unregistered medical image in space to generate a registered medical image corresponding to the unregistered medical image.

3. The system of claim 1, wherein the deep learning model is configured to generate six predictions for the 3D transformation matrix, the six predictions comprising:
rotations of the points of the unregistered medical image in each of three dimensions; and
translations of the points of the unregistered medical image in each of the three dimensions.

4. The system of claim 1, wherein the 3D transformation matrix is a 4×4 matrix.

5. The system of claim 1, wherein the 3D transformation matrix is a rigid transform that includes a first set of parameters and a second set of parameters for the unregistered medical image, wherein the first set of parameters define a rotation of the unregistered medical image and the second set of parameters define a translation of the unregistered medical image.

6. The system of claim 1, further comprising:
a test component that performs a mutual information test comparing the medical image registered in three-dimensional space to a template image, wherein the test component outputs a result of the mutual information test,
wherein, if the result is below a threshold, the medical image registered in three-dimensional space is rejected or flagged.

7. The system of claim 1, wherein the unregistered medical image is a three-dimensional volume of pixels, and wherein the 3D transformation matrix is calculated for the unregistered medical image to map the unregistered medical image to real world coordinates for transformation by the interpolation component.

8. The system of claim 1, further comprising:
a standardization component that receives the unregistered medical image and analyzes the unregistered medical image to validate criteria, the standardization component adjusting the unregistered medical image if the criteria are not met,
wherein the unregistered medical image is a raw image before processing by the standardization component, and
wherein a standardized raw image is input to the interpolation component.

9. The system of claim 8, wherein the standardization component outputs the standardized raw image as the unregistered medical image to the deep learning model and wherein only the standardized raw image or a raw image is input to the deep learning model, or
wherein the standardized raw image and a template image are input to the deep learning model.

10. (canceled)

11. The system of claim 1, wherein, after the trained machine learning logic is trained, learning from the unregistered medical image input to the deep learning model is disabled.

12. The system of claim 1, wherein the deep learning model includes at least two deep learning models, each of the at least two deep learning models being trained on different training data or each of the at least two deep learning models being weighted differently.

13. The system of claim 1, wherein the deep learning model is trained based on a data set of medical images that are manually aligned with a template image, wherein the template image is a specific medical image oriented at a reference position.

14. The system of claim 1, wherein the deep learning model is trained based on a data set of negative correlation data.

15. A method for registering at least one medical image, the method comprising:
receiving a raw medical image;
inputting the raw medical image into a deep learning model; and
generating a 3D transformation matrix for the raw medical image based on trained machine learning logic,
wherein the 3D transformation matrix includes rotation and/or shift parameters that rotate and/or translate points in the raw medical image into a registered orientation.

16. The method of claim 15, further comprising:
outputting the 3D transformation matrix to an interpolation component; and
registering the raw medical image, via the interpolation component, based on the 3D transformation matrix, the registering comprising:
receiving the 3D transformation matrix from the deep learning model;
receiving the raw medical image; and
applying the 3D transformation matrix in three dimensions to rotate and/or translate points of the raw medical image in space to generate a registered medical image corresponding to the raw medical image.

17. The method of claim 16, wherein the 3D transformation matrix is a rigid transform that includes a first set of parameters and a second set of parameters for the raw medical image, wherein the first set of parameters define a rotation for the raw medical image and the second set of parameters define a shift for the raw medical image.

18. The method of claim 16, further comprising;
comparing, in a test component, the registered medical image to a template image via a mutual information test, and
outputting a result of the mutual information test,
wherein, if the result is below a threshold, the raw medical image registered in three-dimensional space is rejected or flagged.

19. The method of claim 16, wherein the raw medical image is a three-dimensional volume of pixels, and wherein the 3D transformation matrix is calculated for the raw medical image to map the raw medical image to real world coordinates for transformation by the interpolation component.

20. The method of claim 16, wherein a standardization component receives the raw medical image and analyzes the raw medical image to validate criteria, the standardization component adjusting the raw medical image if the criteria are not met,
wherein a standardized raw image is input to the interpolation component and the deep learning model.

21. (canceled)

22. (canceled)

23. The method of claim 16, wherein the deep learning model is configured to generate six predictions for the 3D transformation matrix comprising:
rotations of the points of the raw medical image in each of three dimensions; and
translations of the points of the raw medical image in each of the three dimensions.

24. (canceled)

25. (canceled)

26. (canceled)
